(19) United States
(12) Patent Application Publication
(10) Pub. No.: US 2004/0142000 A1
(43) Pub. Date: Jul. 22, 2004

(54) Immune activator

(75) Inventors: Tetsuya Suga, Tokyo (JP); Yoshiaki Ogasawara, Tokyo (JP); Yutaro Kaneko, Tokyo (JP); Masatoshi Kajiura, Kawasaki-shi (JP); Yasuyo Suga, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/692,684

(22) Filed: Oct. 27, 2003

Related U.S. Application Data
(63) Continuation of application No. PCT/JP02/04205, filed on Apr. 26, 2002.

(30) Foreign Application Priority Data
Apr. 27, 2001 (JP) 2001-132513

(51) Int. Cl. A61K 31/715; A61K 35/84

(52) U.S. Cl. 424/195.15; 514/54

(57) ABSTRACT

The present invention relates to an immune activator composition containing either β-glucan or a component derived from a mushroom (e.g., a shiitake mushroom). The invention further provides a method of treating a subject in need thereof by administering the immune activator composition thereto. Specifically, the present invention provides a method of activating immunity or a method of regulating immunity by administering superfine particles of the immune activator composition to a subject in need thereof.

### CROSS-REFERENCE TO RELATED APPLICATIONS

The present application is a continuation of PCT International Application PCT/JP02/04205, filed on Apr. 26, 2002, and claims priority to Japanese Patent Application No. JP 2001-132513, filed on Apr. 27, 2001, each of which is hereby incorporated by reference in their entirety.

### BACKGROUND OF THE INVENTION

1. Field of the Invention

The present invention relates to an immune activator composition containing either β-glucan or a component derived from a mushroom (e.g., a shiitake mushroom). The invention further provides a method of treating a subject in need thereof by administering the immune activator composition thereto. Specifically, the present invention provides a method of activating immunity or a method of regulating immunity by administering superfine particles of the immune activator composition to a subject in need thereof.

2. Discussion of the Background

Mushrooms or components thereof contain various pharmaceutically efficacious components, and thus health foods containing processed powder of a certain mushroom or a hot-water extract thereof are known. In the conventionally known products, however, their various components are not sufficiently utilized, and often the components are not identified.

Accordingly, it is expected that various components of mushrooms or similar materials are utilized effectively much more in the form of pharmaceutical preparations, health foods or functional foods in comparison to the conventional products, in order to maintain and improve the health of animals, particularly humans in daily life, or used in pharmaceutical preparations to treat or ameliorate diseases.

### SUMMARY OF THE INVENTION

It is an object of the present invention to provide pharmaceutical composition for preventing, ameliorating, progress blocking, or therapeutically treating one or more immune-disorder and a method of preventing, ameliorating, progress blocking, or therapeutically treating one or more stress-induced diseases by administering the same.

Specifically, the present invention provides novel superfine particles of a mushroom-derived component such as novel superfine particles of a mushroom extract etc. (preferably, superfine particles of a component obtained by extraction from a mushroom with water) or novel superfine particles of β-glucan. The present invention also provides a composition comprising the novel superfine particles (dispersion of the superfine particles, etc.), an immune activator and/or an immune regulator comprising the superfine particles or the composition as an active ingredient (immune activator/immune regulator), a pharmaceutical composition (particularly, pharmaceutical preparations for diseases starting (occurring) due to abnormalities in immune functions. In this regard, the pharmaceutical preparations includes an antitumor agent, an anti-infective agent, an antiviral agent, an anti-autoimmune disease agent, an anti-diabetes agent and an anti-allergy agent, as well as pharmaceutical preparations for digestive organ diseases (therapeutic agents for irritable bowel syndrome (IBS), inflammatory bowel disease (IBD), constipation, diarrhea and the like)), food and drink (health foods, functional foods etc.), and a process for producing the superfine particles usable as an active ingredient for these diseases or a composition comprising the superfine particles.

The immune activator/immune regulator of the present invention is used in various forms such as a pharmaceutical preparation (pharmaceutical composition), food and drink (health foods, functional foods etc.) etc., and is useful for treatment, amelioration, and prevention from progression, of diseases particularly by activating or regulating immune functions, for prophylaxis of other diseases occurring due to abnormalities in immune functions in (for) patients and the like, and for prophylaxis of various diseases accompanying abnormalities in immune functions for healthy persons by activating or regulating immune functions, and for amelioration of light diseases by improving immune functions therefor, and the like.

The present invention also encompasses a method of activating immunity or a method of regulating immunity, which is useful for treatment (medical treatment), amelioration, prevention from progression and prophylaxis, etc. of tumors, infections, viral infections (diseases), autoimmune diseases, diabetes, allergic diseases, and digestive organ diseases (irritable bowel syndrome (IBS), inflammatory bowel disease (IBD), constipation, diarrhea and the like) and a use of the novel superfine particles as an active ingredient for the immune (immunity) activator, the immune (immunity) regulator, and various chemicals (agents), and for production of foods, drinks, etc. and further pharmaceutical preparations, and the like.

The object of the present invention is to provide food and drink (health foods, functional foods etc.) or a pharmaceutical preparation (pharmaceutical composition), which can be prepared by easy preparative means and effectively utilize various components particularly pharmaceutically efficacious components in mushrooms or similar materials.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 depicts the particle size distribution of a shiitake extract and a micellar shiitake extract in Example 1. In the treatment of the shiitake extract with an emulsifier (emulsifying agent) [conversion into micelles] in Example 1, the particle size distributions of the shiitake extract (left: 1-a) and the micellar shiitake extract (product of the present invention) (right: 1-b) are shown. The particle diameter (μm) is shown on the abscissa, the frequency percentage (%) of the particles for the bar graph on the ordinate (left), and the cumulative frequency percentage (%) of the particles for the curve on the ordinate (right).

FIG. 2 depicts the particle size distribution of a β-glucan solution and micellar β-glucan in Example 2. In the treatment of the β-glucan with an emulsifier (emulsifying agent) [conversion into micelles] in Example 2, the particle size distributions of the β-glucan solution (left: 2-a) and the micellar β-glucan (product of the present invention) (right: 2-b) are shown. The particle diameter (μm) is shown on the abscissa, the frequency percentage (%) of the particles for the bar graph on the ordinate (left), and the cumulative frequency percentage (%) of the particles for the curve on the ordinate (right).

### DETAILED DESCRIPTION OF THE INVENTION

Unless specifically defined, all technical and scientific terms used herein have the same meaning as commonly understood by a skilled artisan in biochemistry, cellular biology, molecular biology, the veterinary sciences, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention is based, in part, on the inventor's discovery that the various components in conventional products obtained from mushrooms were not sufficiently absorbed into animal bodies, and were thus not so utilized as to be expected in the bodies.

As a result of further investigation, the inventors found that when components derived from a mushroom, particularly an extract of a mushroom with water, preferably an extract thereof with hot water, were further finely pulverized to prepare superfine particles and dispersed, for example in water such that an average particle diameter of the superfine particles was 10 μm or less, more preferably 1 μm or less, still more preferably 0.01 to 1 μm in a micellar state, incorporation thereof through mucosa was significantly improved, and as a result, immune functions could be activated or regulated.

Further, the inventors found that the same activity and action were present in superfine particles of β-glucan (including mushroom-derived and non-mushroom derived β-glucan).

The present inventors have found that the above indicated embodiments are particularly useful for stimulating mucosal immunity and are readily incorporated through mucosa (particularly the small intestine) into the body (leading to activation of systemic immunity). As a result, an antitumor effect or a therapeutic treating and/or ameliorating effect on infectious diseases with viruses, such as AIDS, and bacteria or the like, can be expected. Accordingly, the superfine particles can be used as an immune activator/immune regulator (which is an immune activator and/or an immune regulator), and use thereof in the form of pharmaceutical composition(s) or food(s) and drink(s) (which is/are foods(s) and/or drink(s)) (health foods etc.) can be expected.

In an embodiment of the present invention are superfine particles comprising a component selected from a component derived from a mushroom and β-glucan that have been converted into superfine particles, for example superfine particles comprising a component derived from a mushroom or β-glucan is converted into superfine particles. As the prior art relating to the present invention, there is β-glucan encapsulated in liposomes (see International Patent Publication WO 01/85141), but in the present invention, a component selected from a component derived from a mushroom and β-glucan may be in the form of superfine particles, and thus the present invention is evidently different from this prior art in that the β-glucan preparation can be produced by a simpler process without requiring procedures required in the prior art, such as the procedure of encapsulating β-glucan into liposomes and the procedure of separating, from the liposomes, β-glucan not encapsulated in the liposomes by gel permeation column chromatography etc. Accordingly, the present invention does not encompass the form of liposome characterized in that the active ingredient β-glucan is encapsulated therein.

In the present invention, the “component derived from a mushroom” refers collectively to component(s) produced by, in and/or from mushroom(s).

The component derived from a mushroom is not particularly limited insofar as it is a component contained in, or produced by and/or from, a mushroom. For example, the component derived from a mushroom may be an extract containing plural kinds of components contained in a mushroom and/or mushrooms, such as a mushroom extract or an extract of a mushroom with water. Further, the component derived from a mushroom may be a culture product produced in a culture solution by methods of culturing mycelia as described in Japanese Patent Publications JP-B-42-12000, JP-B-46-37873, JP-A-10-287584 etc. The component derived from a mushroom may be a single component such as β-glucan. It may also be a material containing β-glucan.

Although the β-glucan used in the present invention may be a component derived from a mushroom as described above, it may also be a β-glucan not contained in a component derived from a mushroom. The β-glucan not contained in the component derived from a mushroom includes β-glucan as a component derived from e.g. yeast(s) (beer yeast etc.), a component derived from fungi, a component derived from bacteria, a component derived from plant(s) etc.

The extract of a mushroom is preferably a water extract of a mushroom, including extracts with water, hot water, a water-containing solution, etc., in order to obtain a larger amount of the active (effective) ingredient in the present invention. The water extract of a mushroom may be component(s) extracted from a mushroom with water or a material containing the component(s), and the extract includes, for example, a filtrate obtained by filtrating a water extract of a mushroom through a filter paper. The type of filter paper used can be selected as necessary without particular limitation. The extract may further contain components in a from of solid(s) or an aqueous solution, etc., and a dispersion containing, in the filtrate, a part of the water-extracted component(s) in the form of dispersed fine particles (e.g. precipitates of aggregates each having a particle diameter of 100 μm or more, etc.). Thereafter, such a water extract of a mushroom can be subjected to the step of superfine pulverization in the present invention for superfine particles preparation. The aqueous solution containing these components is referred to as an aqueous solution containing a water extract of a mushroom, but it is not always necessary for these components to be completely dissolved in the aqueous solution.

β-glucan used as the other starting material subjected to the step of superfine pulverization may be a β-glucan extract (extracted β-glucan component(s)) as is the case with the mushroom extract described above. Extraction of β-glucan can be performed by extraction methods known in the art, for example a method of extraction with water or hot water (see Biol. Pharm. Bull., 23(7), 866, 2000), a method of treatment with an enzyme (see Japanese Patent Kokai Publications JP-A-5-268905, JP-A-10-287584 etc.) etc. The aqueous solution containing β-glucan extract is referred to as an aqueous β-glucan extract-containing solution, but it is not always necessary for the β-glucan extract to be completely dissolved in the aqueous solution.

In the case of superfine pulverization of a component selected from a component derived from a mushroom and β-glucan, the component preferably forms an aggregate in an aqueous solution. More preferably, the aggregate has a particle diameter of at least 50 μm (50 μm or more).

The “superfine particles” in the present invention have an average particle diameter of preferably 10 μm or less, more preferably 1 μm or less, still more preferably 0.01 to 1 μm or so as determined after being dispersed in water. This average particle diameter can be readily determined with a particle size distribution meter.

Some examples of such superfine particles are as follows:

(1) Superfine particles in a state treated or dispersed with a dispersant preferably in an aqueous solution, which are obtained from a component selected from a component derived from a mushroom and β-glucan.

(2) Superfine particles comprising particles having an average particle diameter of 10 μm or less, which can be obtained upon mixing a dispersant with an aqueous solution containing a component selected from a component derived from a mushroom and β-glucan.

(3) The superfine particles described in the above-mentioned (1) or (2), which comprise particles having an average particle diameter of 1 μm or less obtainable or obtained by a finely pulverizing treatment.

(4) The superfine particles described in the above-mentioned (3), wherein the average particle diameter is 0.01 to 1 μm.

(5) The superfine particles described in any of the above-mentioned (1) to (4), wherein the aqueous solution containing a component selected from a component derived from a mushroom and β-glucan is an aqueous mushroom extract-containing solution obtained by filtration, through a filter paper etc., of an extract (solution) of a mushroom with water or hot water.

(6) The superfine particles described in the above-mentioned (5), wherein the aqueous mushroom extract-containing solution is an aqueous solution containing aggregates obtained by filtering an extract (solution) of a mushroom with water or hot water through a paper filter etc. and then concentrating and/or cooling the filtrate.

(7) The superfine particles described in any of the above-mentioned (1) to (6), wherein the aqueous solution containing a component selected from a component derived from a mushroom and β-glucan is an aqueous solution of β-glucan or an aqueous solution containing β-glucan.

As stated above, the β-glucan may be a component contained in components derived from a mushroom, but is not limited thereto.

The aqueous solution containing a component selected from a component derived from a mushroom and β-glucan is not particularly limited insofar as it is an aqueous solution containing a component selected from a component derived from a mushroom and β-glucan. Furthermore, it is not necessary that the component be dissolved completely in the aqueous solution. For example, aqueous solution containing the component may be an aqueous mushroom extract-containing solution or an aqueous solution containing a water extract of a mushroom or may be a culture solution containing culture products of mushroom mycelia. The aqueous solution is not particularly limited to a solution containing only water other than a component derived from a mushroom, but thus the solution may contain another component in addition to water and the component selected from a component derived from a mushroom and β-glucan. The aqueous solution containing β-glucan (component) is not particularly limited either, and may be an aqueous solution containing β-glucan, and it is not necessary for β-glucan to be completely dissolved in the aqueous solution, and an aqueous β-glucan-containing solution can be used. As described above, this β-glucan may be a component derived from a mushroom or may not be the one contained in components derived from a mushroom. Accordingly, the β-glucan extract component described above can be used therefor.

As to a quantity of dispersant, a dispersant can be mixed with the aqueous solution containing the component(s) (or total components) selected from a component derived from a mushroom and β-glucan such that the weight ratio of the dispersant to the whole sugar (1) (total amount of sugar) contained in the aqueous solution containing the component(s) selected from a component derived from a mushroom and β-glucan is preferably at most 100 (100 or less), more preferably 10 or less, still more preferably 0.05 to 5 or so.

The content of a component selected from a component derived from a mushroom and β-glucan in the aqueous solution is not particularly limited, but it is preferable for solubility that the content of the component(s) (or total components) selected from a component derived from a mushroom and β-glucan in the aqueous solution can be determined such that the concentration of the whole sugar is at most 50 mg/ml (not higher than 50 mg/ml), more preferably in the range of 0.5 to 50 mg/ml or so.

The identity of the dispersant is not particularly limited, and examples of the dispersant include surfactants, polymers, sugars, sugar alcohols, glycerides, acids, bases, salts, etc. Among these, an emulsifier as a typical example of the surfactants is preferable, and lecithin can be used more preferably.

The superfine particles of the present invention can be obtained and used in the state of micelles with an emulsifier.

Although the method of finely pulverizing treatment is not particularly limited, a wet milling process with a high-pressure emulsifier (homogenizer), a media mill, supersonic waves (sonicator) etc. is preferable. For example, the high-pressure emulsifier can be used to prepare superfine particles of 1 μm or less described above. The emulsifying pressure used is preferably at least 300 kgf/cm², more preferably at least 500 kgf/cm², still more preferably at least 800 kgf/cm², and the emulsifying pressure can be reduced by increasing the number of times the treatment was repeated.

The superfine particles can be obtained by filtering preferably an extract (solution) of water or hot-water mushroom or an extract solution of β-glucan through a filter paper etc. to give an aqueous solution containing a mushroom extract or β-glucan extract, mixing a dispersant therewith preferably under stirring. Accordingly, the superfine particles containing particles having an average particle diameter of 10 μm or less can be obtained in this manner.

In this case, it is possible to use, for example, not only an aqueous solution containing aggregates obtained by filtering a water or hot-water extract of a mushroom as the aqueous mushroom extract-containing solution (the aqueous solution containing a mushroom extract) through e.g. a filter paper etc. and then concentrating and/or cooling the filtrate, but also the aqueous filtrate which will, upon being concentrated and/or cooled, give the aggregates. The β-glucan can also be used by preparing the same aqueous solution as described above by the same treatment as above.

For use as an immune activator or an immune regulator, the superfine particles can be used in the form of an aqueous solution or dispersion in which the active ingredient is finely pulverized for example (mushroom extract treated with a dispersant as described above, etc.), desirably in the form of micelles. Among these, the mushroom extract or β-glucan treated with a dispersant can be conveniently used. Thus, the superfine particles in such various forms also fall under the scope of the superfine particles of the present invention.

The extract with hot water, i.e., the extract of a mushroom with hot water, is preferably (from the viewpoint of efficient extraction of the active/effective ingredient with hot water) from a mushroom after milling.

The superfine particles can be absorbed or incorporated (ingested) through mucosa in small intestines of animals particularly humans, thus exhibiting an immune activating effect or an immune regulating effect.

Another embodiment of the present invention lies in an immune activator and/or an immune regulator characterized by comprising any superfine particles described above (immune activator/immune regulator).

In still another embodiment of the present invention is a pharmaceutical composition comprising any superfine particles described above (agent (medicine); pharmaceutical preparation). The pharmaceutical composition may contain a pharmaceutically acceptable carrier, excipient (bulk filler) (or diluent) etc.

The immune (immunity) activator/immune regulator can also be used in the form of food and drink (food and/or drink).

Examples of the above medicine include an antitumor agent, an anti-infective agent, an antiviral agent, an anti-autoimmune disease agent, an anti-diabetes agent and an anti-allergy agent, as well as pharmaceutical preparations for digestive organ diseases (therapeutic agents for irritable bowel syndrome (IBS), inflammatory bowel disease (IBD), constipation, diarrhea and the like).

Another embodiment of the present invention is food and drink (food and/or drink) comprising any superfine particles described above (superfine particles of the present invention). The content of the superfine particles is not limited, and the content of the superfine particles in food and drink such as health foods is preferably about (approximately) 0.01 to 80% by weight, more preferably about (approximately) 0.05 to 20% by weight in terms of the whole sugar thereof.

The food and drink of the present invention can be used as health foods, functional foods, health drinks, functional drinks etc. The food and drink are particularly suitable for patients with diseases such as cancers, microbial infectious diseases, viral infectious diseases, autoimmune diseases, diabetes, allergic diseases, and digestive organ diseases (irritable bowel syndrome (IBS), inflammatory bowel disease (IBD), constipation, diarrhea and the like).

In still another embodiment of the present invention is a superfine particle(s)-containing composition characterized by comprising an aqueous solution having the superfine particles of the present invention dispersed therein, that is, an aqueous solution (dispersion etc.) comprising the superfine particles.

The dispersion can be used in a pharmaceutical composition (pharmaceutical preparation) in the same manner as described above. In this case, the pharmaceutical composition can contain a pharmaceutically acceptable carrier or excipient (bulking filler).

The dispersion can be used in food and drink (food and/or drink) in the same manner as described above. In this case, the food and drink (food and/or drink) can contain (comprise) the composition in an amount of 0.05 to 5% by weight in terms of (based on) the whole sugar thereof.

The food and drink of the present invention can be used as health foods, functional foods, health drinks, functional drinks etc. The food and drink are particularly suitable for patients with diseases such as cancers, microbial infectious diseases, viral infectious diseases, autoimmune diseases, diabetes, allergic diseases, and digestive organ diseases (irritable bowel syndrome (IBS), inflammatory bowel disease (IBD), constipation, diarrhea and the like).

With respect to the content of the components in the composition, the composition can contain sugar(s) in an amount of preferably 1 to 20000 mg, more preferably 10 to 1000 mg and dispersant(s) in an amount of preferably 1 to 20000 mg, more preferably 10 to 1000 mg per 100 g of the composition.

The superfine particle(s)-containing composition is not particularly limited insofar as it is a composition comprising the superfine particles of a component derived from a mushroom, β-glucan etc. The composition includes preferably the aqueous solution containing the superfine particles of a component derived from a mushroom or β-glucan and a dispersant, more preferably the superfine particles prepared by mixing the aqueous solution containing the component(s) with a dispersant, the aqueous solution wherein the superfine particles are dispersed and the like.

Another embodiment of the present invention is a process for producing the superfine particles characterized in that a component selected from a component derived from a mushroom and β-glucan, that is, a component derived from a mushroom or β-glucan, is subjected to a step of superfine pulverization, for example, a process for producing superfine particles which comprises subjecting a mushroom to a step of extraction with water and then subjecting the resulting aqueous extract to a step of superfine pulverization.

In particular, the superfine particles containing particles having an average particle diameter of 10 μm or less can be produced by filtering a water or hot-water extract of a mushroom through a filter paper etc. to give an aqueous mushroom extract-containing solution (an aqueous solution containing an extract of a mushroom) and then mixing a dispersant therewith preferably under stirring. In this case, it is possible to use not only an aqueous solution containing aggregates obtained by filtering a water or hot-water extract of a mushroom as the aqueous mushroom extract-containing solution (aqueous solution containing the extract thereof) through e.g. a filter paper and then concentrating and/or cooling the filtrate, but also the aqueous filtrate as it is from the filtration which will, upon being concentrated and/or cooled, give the aggregates, as described above. In case of the α-glucan, the desired particles can also be prepared in the same manner as described above.

The step of superfine pulverization can comprise a step of preparing particles having an average particle diameter of 10 μm or less by mixing a dispersant with an aqueous solution containing a component selected from a component derived from a mushroom and β-glucan, for example with an aqueous solution containing a component derived from a mushroom, and the step of superfine pulverization can comprise a step of preparing particles having an average particle diameter of 1 μm or less by finely pulverizing treatment step, for example a step of treatment with (by) a high-pressure emulsifier.

By this method, the superfine particles preferably having an immune activating activity and/or an immune regulating activity can be obtained.

A further embodiment of the present invention is a process for producing a composition comprising (containing) the superfine particles, characterized by subjecting a component selected from a component derived from a mushroom and β-glucan, for example a component derived from a mushroom, to a step of superfine pulverization.

This process can be carried out according to the process for producing the superfine particles described above. The step of superfine pulverization can similarly use a step of preparing particles having an average particle diameter of 10 μm or less by mixing a dispersant with an aqueous solution containing a component selected from a component derived from a mushroom and β-glucan or a step of preparing particles having an average particle diameter of 1 μm or less by finely pulverizing treatment step, for example a step of treatment with (by) a high-pressure emulsifier. Further, the component selected from a component derived from a mushroom and β-glucan may be a water or hot water extract obtained by subjecting a mushroom to a step of extraction with water or hot water, and the aqueous solution containing a mushroom-derived component may be an aqueous solution containing a mushroom extract (an extract of a mushroom) obtained by filtering a water or hot water extract of a mushroom thorough a filter paper etc. On one hand, in case of the β-glucan extract it can also be prepared in an analogous manner.

By this method, the dispersion preferably having an immune activating activity and/or an immune regulating activity and the like can be obtained.

The average particle diameter in the methods described above refers to the average particle diameter of the particles measured (determined) in the form of a dispersion in water as described above.

Another embodiment of the present invention is a method of activating immunity or a method of regulating immunity characterized by ingesting or administering the superfine particles of the present invention into a living body, and is extremely useful for treatment, amelioration, prevention from enlargement, prophylaxis, etc. of diseases such as tumors, infectious disease, viral infections, autoimmune diseases, diabetes, allergic diseases, and digestive organ diseases (irritable bowel syndrome (IBS), inflammatory bowel disease (IBD), constipation, diarrhea and the like) and the like.

In the ingestion or administration forms, it is possible to use the immune activator and the immune regulator etc.; or the antitumor agent, anti-infective agent, antiviral agent, anti-autoimmune disease agent, anti-diabetes agent and anti-allergy agent, as well as pharmaceutical preparations for digestive organ diseases (therapeutic agents for irritable bowel syndrome (IBS), inflammatory bowel disease (IBD), constipation, diarrhea and the like) and the like, as described above. In particular, the superfine particles can be used preferably in the form of the pharmaceutical composition and the food and drink (food and/or drink) described above.

In another embodiment of the present invention is a use of the novel superfine particles in (for) the immune activator or the immune regulator or production thereof; or a use of the superfine particles in (for) the antitumor agent, anti-infective agent, antiviral agent, anti-autoimmune disease agent, anti-diabetes agent and anti-allergy agent, as well as pharmaceutical preparations for digestive organ diseases (therapeutic agents for irritable bowel syndrome (IBS), inflammatory bowel disease (IBD), constipation, diarrhea and the like) and the like, and further in a use of the superfine particles in (for) production of pharmaceutical preparations.

The immune activator and the immune regulator, or the antitumor agent, anti-infective agent, antiviral agent, anti-autoimmune disease agent, anti-diabetes agent and anti-allergy agent, as well as pharmaceutical preparations for digestive organ diseases (therapeutic agents for irritable bowel syndrome (IBS), inflammatory bowel disease (IBD), constipation, diarrhea and the like) and the like are as described above, and preferable examples thereof include the form of the pharmaceutical composition or the form used for the food and drink (food and/or drink), as described above.

Hereinafter, the mode for carrying out the invention is described in more detail. Preferable and typical examples of the present invention are mainly described, but the present invention is not limited to such preferable and typical examples.

Superfine Particles; Superfine Particles of a Mushroom Extract, a β-Glucan Extract

First, the superfine particles of the present invention are described by referring mainly to the production of the superfine particles of a mushroom extract.

In the present invention, the type of mushroom is not particularly limited. Further, the site used in extraction is not particularly limited either. Edible mushrooms can be used. Typical examples include, but are not limited to, the followings.

The mushroom in the present invention refers to fungi capable of forming fruit body.

Lentinus edodes

Pleurotus ostreatus

Pholiota nameko

Flammulina velutipes

Tricholoma matsutake

Lyophyllum shimeji

Schizophyllum commune

Crepidotus variabilis

Lyophyllum ulmarium

Grifola umbellata

G. frondosa

Coriolus versicolor

Fomes fomentarius

Volvavella volvacea

Auricularia aurcula-judae

Ganoderma lucidum

G. applanatum

Fomitopsis pinicola

Dictyophora indusiata

Sparassis crispa

Agaricus blazei

Peziza vesiculosa

The site of the mushroom that is used is not particularly limited to special regions such as fruit body, mycelium etc. as described above. The components of a raw mushroom are varied depending on the type of mushroom, and for example, a shiitake fruit body is composed of about 90% (by weight) water, about 5% (by weight) sugar, about 2% (by weight) protein, about 1% (by weight) fiber and about 2% (by weight) other components. Accordingly, the active (efffective) ingredient in the present invention is superfine particles of non-water components extracted with water (hot water etc.).

On one hand, β-glucan is not particularly limited either, and may be a component derived from a mushroom, a component derived from yeasts, a component derived from fungi, a component derived from bacterium (bacteria), a component derived from plant(s), etc.

It is not particularly difficult to obtain an extract of a mushroom-derived component or β-glucan. For example, a mushroom may be used in extraction with water such as hot water etc. In the case, the extraction step can be easily carried out by subjecting its milled material to the step of extraction with hot water. When hot water is used, a temperature of about 60 to 100° C. or so is used. A filtrate obtained by a filter paper (filter paper can be selected as necessary without particular limitation to its type) etc. after the extraction step, even whether the filtrate is a suspension containing finely pulverized particles or a solution containing aggregates obtained by further concentrating, cooling and the like the suspension, falls under the scope of the extract in the present invention.

The extract in the present invention may be a component (which may not necessarily be completely dissolved) contained in water in the extraction step with water (hot water etc.) described above, and therefore, the filtrate obtained by filtration through a filter paper etc. after the extraction step, and the fine particle component (aggregates) coagulated from the extract solution by concentration, cooling etc. also fall under the scope of this extract.

As the extracting solvent, it is possible to use not only water but also other organic solvents. However, as the extracting solvent, water alone or a mixed solution of water and a small amount of an organic solvent is preferably used. As a matter of course, the extraction with such a water-containing solution also falls under the scope of the water extraction in the present invention. Further, even if an acid, an alkali or an inorganic substance is contained in the extracting solvent or added thereto if necessary in such a range that the amount of a mushroom extract is not adversely affected, there is no problem.

By further subjecting the extracted component to a step of superfine pulverization, the superfine particles having an immune activating activity or an immune regulating activity can be produced.

Hereinafter, the production of the superfine particles of the present invention is described in more detail by reference to preferable examples.

In an extract of a mushroom with hot water etc., for example in an extract obtained by extraction, then filtering the hot extract (filtration through Celite etc.) and cooling the filtrate or concentrating and then cooling the filtrate, aggregates having an average particle diameter of 100 μm or more are coagulated. The aggregates are considered to be those formed by aggregation of polysaccharides such as β-glucan or peptidoglycan etc. in the extract. For example, when a filtrate obtained by extracting from a milled raw shiitake mushroom at 95° C. for 3 to 15 hours and filtering the extract through Celite is observed, the filtrate was confirmed to be a suspension having finely pulverized particles dispersed in the filtrate. By measuring the particle diameter of this particle, it was also confirmed that the particle is an aggregate having a median diameter of about 250 μm, and the components of this particle are β-glucan, peptidoglycan etc.

When such an extract (extract containing aggregates each having an average particle diameter of 100 μm or more) is orally ingested or administered, the active (effective) ingredient in the extract is not efficiently absorbed through a mucosa in the intestinal tract and is thus not effectively utilized in the living body. According to the superfine particles of the present invention, on the other hand, the active ingredient in the extract can be efficiently absorbed or incorporated through a mucosa in the intestinal tract to induce or cause immune reaction in lamina propria mucosae.

That is, the mushroom extract solution containing aggregates obtained by filtering a hot extract of a mushroom with water (hot water etc.) and then cooling the filtrate or concentrating and cooling the filtrate is dispersed with a dispersant etc. to disperse the coagulated aggregates, whereby superfine particles having an average particle diameter minimized to preferably 10 μm or less, more preferably 10 μm or less, still more preferably 0.01 to 1 μm or so can be produced.

For superfine pulverization of the active ingredient or coagulated aggregates in the mushroom extract, a dispersant can be used in a solution containing the active ingredient of the mushroom extract contained therein, thus dispersing the coagulated aggregates, or the active ingredient of the mushroom extract contained therein can be embedded in microcapsules etc., or the coagulated aggregates can be dispersed with a dispersant and then embedded in microcapsules etc.

Whether an immune activating action is present or not can be easily confirmed by measuring an antitumor activity, an NK (natural killer) activity, delayed type hypersensitive reaction, an amount of intracellular and extracellular cytokines, an amount of antibody produced, or the like.

The method of superfine pulverization is not particularly difficult, and superfine pulverization can be effected by using, for example, a stirrer or a homogenizer and a suitable dispersant. Further, superfine pulverization can also be effected by finely pulverizing treatment with a high-pressure emulsifier, a medium mill, supersonic waves etc.

When a dispersant is used, the dispersant is not particularly limited insofar as it is a dispersant capable of dispersing the particles in a solution, and examples thereof include surfactants, polymers, sugars, sugar alcohols, glycerides, acids, bases, salts etc. The substances used also as the emulsifier (emulsifying agent) are preferably used therefor. The emulsifiers used therefor are more preferably edible emulsifiers such as lecithin, lysolecithin, bile acid etc. In the present specification (description), dispersion with an emulsifier (emulsifying agent) refers particularly to conversion into micelles (micelles formation), but the present invention is not limited thereto, and conversion into micelles with a dispersant other than an emulsifier also falls under the scope of the present invention.

The finely pulverizing step in the present invention may be carried out at any time(s) before, after and during a step for obtaining the active ingredient such as, for example, the extraction step.

When the superfine particles of the present invention are measured in the form of a dispersion in water, the superfine particles having an average particle diameter of preferably 10 μm or less, more preferably 1 μm or less, still more preferably 0.01 to 1 μm or so can be used. When used as an immune activator/immune regulator, a solution (dispersed solution) of the superfine particles treated with a dispersant, particularly a micellar solution treated with an emulsifier (emulsifying agent), is preferably used for digestion and incorporation, but the superfine particles in a dried state can also be used as an immune activator/immune regulator.

In the present invention, the method of measuring (determining) the superfine particles can be carried out by utilizing a method of measuring usual particles, particularly dispersed particles. For example, the superfine particles can be measured by a laser diffraction/scattering particle size distribution measuring method using a particle size distribution meter.

Immune Activator/Immune Regulator

As described above to some degree, the superfine particles of the present invention can be utilized as the active ingredient of an immune activator/immune regulator (immune activator/immune regulator of the present invention). A carrier or an excipient (bulking filler) (or a diluent) usable in the pharmaceutical composition or the food and drink (food and/or drink) according to the present invention can also be used. Specifically, the immune activator/immune regulator can be used as the pharmaceutical composition, the food and drink (food and/or drink) (health foods etc.) and the like.

Whether an immune activating action or immune regulating action is present or not can be easily confirmed by measuring e.g. an antitumor activity, an NK activity, delayed type hypersensitive reaction, an amount of intracellular and extracellular cytokines or an amount of antibody produced.

Pharmaceutical Composition

The pharmaceutical composition (agent; drug; pharmaceutical preparation) of the present invention is an agent (a pharmaceutical preparation) which comprises the superfine particles as described above, preferably the solution treated with a dispersant (dispersed solution), more preferably the solution containing the micellar component(s) as the active ingredient with an emulsifier (emulsifying agent) and which can be used for treatment, amelioration and prevention from enlargement, of diseases accompanying abnormalities in immunity or for prophylaxis etc. of other diseases by activating or regulating immunity, particularly systemic immunity. For example, the pharmaceutical composition can be used for an antitumor agent, an anti-infective agent, an antiviral agent, an anti-autoimmune disease agent, an anti-diabetes agent and an anti-allergy agent, as well as an agent for digestive organ diseases (therapeutic agent for irritable bowel syndrome (IBS), inflammatory bowel disease (IBD), constipation, diarrhea and the like) and the like, and used for treatment or prevention (preservation) of these various diseases.

The subject to which this agent (pharmaceutical preparation) is applied is an animal, particularly a human seeking activation or regulation of immunity, particularly systemic immunity.

One characteristic of the pharmaceutical preparation of the present invention is that an excellent effect is brought about even by oral administration; a component derived from a mushroom and β-glucan, for example an extracted mixture from a mushroom, particularly from an edible mushroom etc. can be used; and the pharmaceutical preparation is particularly excellent in safety. Accordingly, the form of administration is not particularly limited. Various forms of administration such as oral administration, parenteral administration (subcutaneous administration, intramuscular administration, nasal administration, aerosol administration etc.) can be used, and the pharmaceutical preparation can be applied widely and easily to patients seeking an immune activating action and/or an immune regulating action. The pharmaceutical preparation is suitable for safety and oral administration, and can thus be used in the form of health foods, functional foods, health drinks, functional drinks etc. described later, in order to prevent and ameliorate the intended disease.

In the present invention, the pharmaceutical preparation can be mixed or combined with other pharmaceutical component(s) (pharmaceutically active substance(s)), and insofar as a certain pharmaceutical preparation comprises the desired active ingredient in the present invention to exhibit the desired pharmacological activity (immune activating activity or immune regulating activity), such pharmaceutical preparation falls under the scope of the pharmaceutical preparation of the present invention.

In addition, the pharmaceutical preparation can further contain a wide variety of pharmacologically acceptable pharmaceutical material(s) (as adjuvant etc.) for pharmaceutical preparation. The pharmaceutical material(s) can be selected suitably depending on the form of the preparation, and examples thereof include excipients, diluents, additives, disintegrating agents, binders, coating agents, lubricants, sliding agents, lubricants (lubricant pharmaceuticals), flavorings, sweeteners, emulsifiers (emulsifying agents), solubilizers etc. Further examples of the pharmaceutical materials include magnesium carbonate, titanium dioxide, lactose, mannitol and other sugars, talc, milk protein, gelatin, starch, cellulose and derivatives thereof, animal and vegetable oils, polyethylene glycol, and solvents such as sterilized water and monovalent or polyvalent alcohols, for example glycerol.

The pharmaceutical preparation of the present invention can be prepared in various pharmaceutical forms known in the art or to be developed in the future as described above, for example in administration forms for oral administration, intraperitoneal administration, transdermal administration, inhalation administration etc. To prepare the agent (pharmaceutical preparation) of the present invention in such various pharmaceutical preparation forms, methods known in the art or to developed in the future can be suitably used.

The forms of these various pharmaceutical preparations include, for example, suitable solid or liquid pharmaceutical forms such as granules, powders, coated tablets, tablets, (micro)capsules, suppositories, syrups, juices, suspensions, emulsions, dropping agents, injection solutions, preparations prolonging release of the active agent, etc.

As a matter of course, the pharmaceutical preparation of the present invention in the pharmaceutical preparation forms illustrated above should contain a pharmaceutically effective amount of the above described component(s).

The amount of the pharmaceutical preparation of the present invention administered is selected suitably depending on the type and severity of the disease, the form of the pharmaceutical preparation, etc. For example, the superfine particles of the active ingredient can be administered orally to a patient in a daily dose of preferably 1 mg to 50 g or so, more preferably 10 mg to 10 g or so, still more preferably 50 mg to 5 g or so expressed in terms of the whole sugar thereof. In the case of a more severe disease-state, the dose can be increased further. With respect to the frequency and intervals of administration, the pharmaceutical preparation of the superfine particles can be administered once every a few days or once every day, but is usually administered for example before, between and/or after meal (or each meal) in 2 to 4 divided portions several times every day. Preferably, the pharmaceutical preparation of the superfine particles is administered before meal. In the case of intravenous administration, the dose may be one tenth to hundredth (1/10 to 1/100) as small as the dose in oral administration.

Food and Drink

Even when the food and drink (food and/or drink) of the present invention are used particularly as health foods or functional foods, the food and drink can be prepared on the basis of the above-described oral preparation by adding component(s) (including extract(s) derived from different mushroom(s)) and additives necessary for health foods or functional foods. In this regard, edible or nutrient ingredients etc. used in food and drink can be added if necessary and used. Usually, the superfine particles can be contained in an amount of preferably 0.01 to 80% or so by weight, more preferably 0.05 to 20% or so by weight in terms of the whole sugar therein.

Flavorings or sweeteners usable in food and drink can be used to form a solution usable in the form of drink or a form in the form of tablets, granules or capsules, or a form in a jelly or ice cream form, or one in a frozen form or the like.

The food and drink can be used for prevention not only for healthy persons but also for patients with severe to light various diseases, particularly for patients seeking systemic immunity activation or immunity regulation without limitation to patients with diseases accompanying abnormalities in immune functions. For animals other than humans, the food and drink can be applied in forms such as feed, pharmaceutical products (preparations) and pharmaceutical compositions.

Superfine Particles-Containing Composition

This invention lies in an aqueous solution wherein the superfine particles of a component selected from a component derived from a mushroom and β-glucan are dispersed, and this invention(s) can be easily understood and practiced from the description for the superfine particles, the various uses thereof or the production method (process) described above. As a matter of course, the composition similar to the superfine particles can be used in various uses, particularly for a pharmaceutical composition or food and drink (food and/or drink), and the above these descriptions therefor can also be applied to this invention.

As described above, a still other aspect of the present invention lies in a method of activating immunity or a method of regulating immunity characterized by ingesting or administering the superfine particles of the present invention into a living body, and a further still other aspect of the present invention lies in a use of the superfine particles for an immune activator or an immune regulator; or uses of the superfine particles for an antitumor agent, anti-infective agent, antiviral agent, anti-autoimmune disease agent, anti-diabetes agent and anti-allergy agent, as well as pharmaceutical preparations for digestive organ diseases (therapeutic agents for irritable bowel syndrome (IBS), inflammatory bowel disease (IBD), constipation, diarrhea and the like) and the like, and further in a use thereof for production of the pharmaceutical products (preparations).

These inventions can be easily carried out on the basis of the above description for the immune activator or the immune regulator, or the descriptions for the antitumor agent, anti-infective agent, antiviral agent, anti-autoimmune disease agent, anti-diabetes agent and anti-allergy agent, as well as pharmaceutical preparations for digestive organ diseases (therapeutic agents for irritable bowel syndrome (IBS), inflammatory bowel disease (IBD), constipation, diarrhea and the like) and the like or the descriptions for the pharmaceutical composition, the food and drink (food and/or drink) etc., or on the basis of preferable embodiments (Examples) described later, or by reference to known techniques if necessary.

This invention (product of the present invention) can be used not only in humans but also in other animals as described above, and for example, the product of the present invention is also useful as feed for animals in livestock industry (cattle, pigs, sheep, horses, birds etc.), pets (dogs, cats etc.) or fishes in fisheries and raising industry (bony fishes, crustaceans etc.) or as additives to be added to feed or as pharmaceutical preparations or pharmaceutical compositions.

Having generally described this invention, a further understanding can be obtained by reference to certain specific examples, which are provided herein for purposes of illustration only, and are not intended to be limiting unless otherwise specified.

### EXAMPLES

### Example 1

Method of Extraction from Shiitake Mushroom

About 4 L of water were added per kg (raw weight) of shiitake mushrooms, and the mushrooms were disrupted using a colloid mill. The volume of the solution after disruption was about 6 L. The resulting solution was boiled at 95° C. for 15 hours while heating under reflux to prevent evaporation of water, and the resulting extract was filtered. The filtrate was concentrated at 60° C. under reduced pressure to give about 1 L concentrate. The sugar content of the extract was analyzed by the phenol-sulfuric acid method, indicating that the concentration of sugars in the extract was a 20 mg/ml (this extract may be referred to hereinafter as “extract of shitake”; or “shitake extract”).

Conversion into Micelles-Treatment of the Shiitake Extract With an Emulsifying Agent

Lecithin (SLP-PC70) manufactured by Tsuru Lecithin Kogyo Co., Ltd. was added to deionized water to prepare a solution containing lecithin at a concentration equivalent to the whole sugar in the shiitake extract (see above). To the lecithin solution an equal volume of the above shiitake extract was added, and the mixture was stirred under vacuum (vacuum pressure: −60 cmHg; number of revolutions (rotation frequency) of an anchor mixer: 50 rpm; number of revolutions of a homomixer: 15,000 rpm) by an Agi homomixer 2M-2 model manufactured by Tokushu Kika Kogyo Co., Ltd., to prepare a preliminary micellar solution. The resulting preliminary emulsified solution was subjected to high-pressure emulsification treatment (emulsification pressure 1,500 kgf/cm²) with a high-pressure emulsifier H11 model in a 2-step handle system, manufactured by Sanwa Kikai Co., Ltd., to prepare a micellar solution of the shiitake extract having a median diameter of about 100 nm (micellar shiitake extract: the product of the present invention). Laser diffraction/scattering particle size distribution measurement method using an LA-910 particle size distribution meter manufactured by Horiba Seisakusho Co., Ltd. was used to measure the median diameter of the particles.

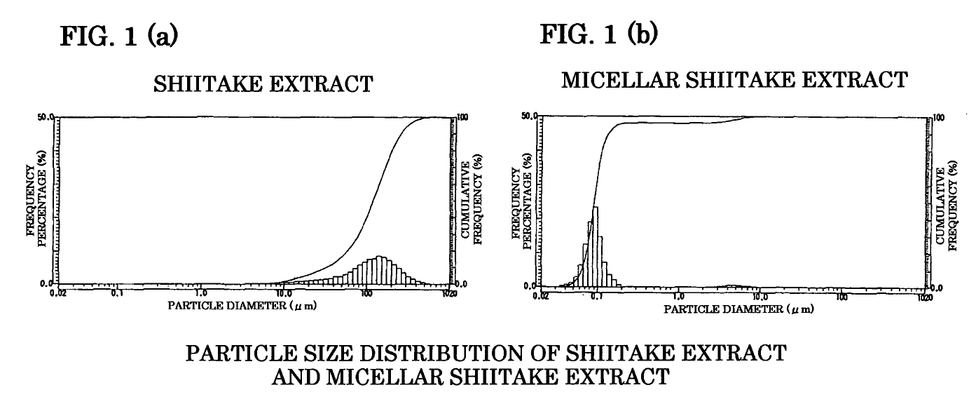

The measurement results of the particle size distribution of the shiitake extract and the micellar shiitake extract are shown in FIG. 1. These results indicate that the component in the shiitake extract had a median particle diameter of about 120 μm, and by treatment of the component in the shiitake extract with an emulsifier (emulsifying agent) (“conversion into micelles”), the component could be converted into superfine particles having a median particle diameter of about 0.09 μm.

### Example 2

Preparation of β-Glucan Solution

Purification of β-glucan from raw shiitake mushrooms was performed according to the method of Chihara et al. (Cancer Res., 30, 2776 (1970)). Specifically, fruit bodies of raw shiitake mushrooms were extracted with hot water, then repeatedly fractionated by precipitation with ethanol, then fractionated by precipitation with cetyltrimethyl ammonium hydroxide, then fractionated by elution with acetic acid and then fractioned by elution with sodium hydroxide followed by removal of protein. By the foregoing method, a white powder of β-glucan was obtained. The resulting white powder was suspended in distilled water, homogenated and subjected to high-temperature and high-pressure treatment (121° C., 20 minutes) in an autoclave to prepare 2 mg/ml β-glucan solution.

Conversion into Micelles-Treatment of the Shiitake Extract With an Emulsifying Agent

Lecithin (SLP-PC70) manufactured by Tsuru Lecithin Kogyo Co., Ltd. was added to deionized water to obtain a solution containing 8 mg/ml lecithin. An equal volume of the above β-glucan solution was added to the lecithin solution, and the mixture was subjected to high-pressure emulsification treatment (emulsification pressure 1,500 kgf/cm²) with a high-pressure emulsifier H11 model in a 2-step handle system, manufactured by Sanwa Kikai Co., Ltd., to prepare a micellar solution of β-glucan having a median diameter of about 100 nm (micellar β-glucan: the product of the present invention). Laser diffraction/scattering particle size distribution measurement method using an LA-910 particle size distribution meter manufactured by Horiba Seisakusho Co., Ltd. was used to measure the median diameter of the particles.

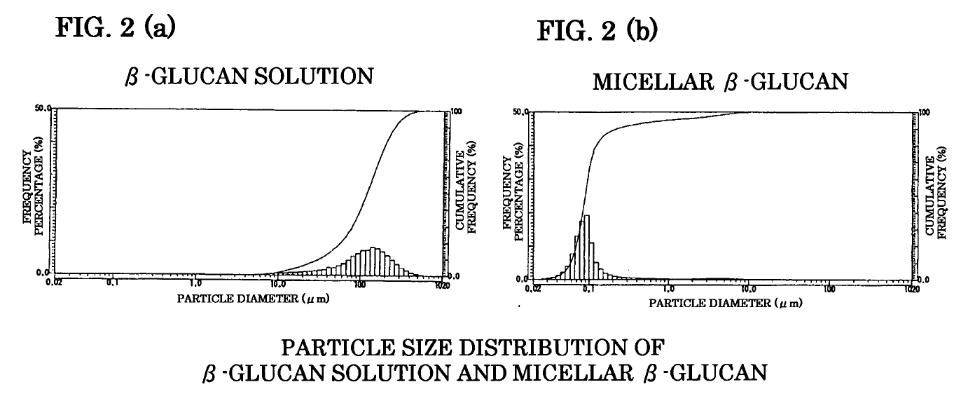

The measurement results of the particle size distribution of the β-glucan solution and the micellar β-glucan are shown in FIG. 2. These results indicate that β-glucan in the β-glucan solution formed aggregates having a median particle diameter of about 120 μm, and by treatment thereof with an emulsifier (emulsifying agent) (“conversion into micelles”), the aggregates could be converted into superfine particles having a median particle diameter of about 0.09 μm.

### Example 3

Test Method-Example Using an S180 Subcutaneous Inoculation Model

Sarcoma 180 tumor cells maintained by intraperitoneal injection in ICR mice (female, 4-week-old) were collected in the form of ascitic fluid and prepared at a density of 3×10⁷ cells/ml with physiological saline. This cell suspension was subcutaneously inoculated in a volume of 0.1 ml/mouse through a 25 G needle into a right groin of ICR mice (female, 4-week-old).

On the next day, the mice were grouped (7 mice/group) depending on their weight, and were identified, and then administrations of the extract of shiitake (shiitake extract) and the micellar solution of the extract of shiitake (micellar shiitake extract) were initiated. The administration was performed orally (one time/day), 5 times per week, and the administration was conducted 10 times in total. The dose for each administration was as follows: the extract (sample) adjusted to a concentration of 1 mg/ml was given in a dose of 0.2 ml/mouse to a 10 mg (in terms of whole sugar)/kg administration group, and the extract (sample) adjusted to 10 mg/ml was given in a dose of 0.2 ml/mouse to a 100 mg (in terms of whole sugar)/kg administration group.

The number in the brackets after “Shiitake extract” and “Micellar shiitake extract”, shown in the item “Administration group” in Tables 1 and 2, is the administration dose of sample in terms of whole sugar (unit: mg/kg). In this example, the particles (the superfine particles) subjected to superfine pulverization treatment with an emulsifier (emulsifying agent) are referred to as the micellar shiitake extract.

The tumor size and the body weight were measured once per week. From the tumor size, the tumor weight was calculated according to the following formula:

Tumor weight (mg)=tumor minimum diameter (mm)²×tumor maximum diameter (mm)÷2

Further, the host weight was also calculated from the tumor weight and body weight.

Host weight (g)=body weight (g)−tumor weight (g)

From the tumor weight, the degree of inhibition of tumor growth was calculated.

Degree of inhibition of tumor growth (%)=(1−tumor weight of administration group÷tumor weight of non-treatment group)×100

From the degree of inhibition of tumor growth in each week and the number of tumor bearing mice on the 35th day after inoculation of the tumor, the pharmaceutical effect on this model was evaluated. The results of the inhibitory effect of the micellar shiitake extract on the tumor growth are shown in Tables 1 and 2.

TABLE 1
Tumor weight (g)

| | Day 16 | | | |
|---|---|---|---|---|
| Administration group | Average (g) | S.E. | t-test | M test |
| Non-treatment group | 2.493 | 0.246 | — | — |
| Emulsifier only | 2.817 | 0.401 | N.S. | N.S. |
| Shiitake extract (10) | 1.844 | 0.363 | N.S. | N.S. |
| Shiitake extract (100) | 2.712 | 0.522 | N.S. | N.S. |
| Micellar shiitake extract | 1.507 | 0.163 | p < 0.01 | p < 0.01 |
| (10) | | | | |
| Micellar shiitake extract | 1.744 | 0.394 | N.S. | N.S. |
| (100) | | | | |

Day 16: 16th day after inoculation of the tumor
t-test: Student's t-test: t-test of each group as compared with the non-treatment group was conducted.
M test: Mannwhitney-U test: Rank test of each group as compared with the non-treatment group was conducted.
N.S., not significant;
p < 0.01, significant.

In the group that was administered the micellar shiitake extract, the tumor growth was inhibited gradually as compared with the non-treatment group during the administration period, and on Day 16 (2nd day after the administration was finished), the tumor growth was inhibited significantly (p<0.01) in the group orally administered the micellar shiitake extract in an administration dose of 10 mg (in terms of whole sugar)/kg.

TABLE 2
Inhibitory effect on tumor growth

| | Degree of inhibition |
|---|---|
| | of tumor growth (%) |
| Administration group | Day 16* |
| Non-treatment group | — |
| Emulsifier only | −13.0 |
| Shiitake extract (10) | 26.0 |
| Shiitake extract (100) | −8.8 |
| Micellar shiitake extract (10) | 39.6 |
| Micellar shiitake extract (100) | 28.8 |

*16th day after transplant of the tumor

As is evident from the results shown above, it was confirmed that the superfine particles of the present invention exhibit the desired pharmaceutical effect as compared with the conventional products.

### Example 4

Quantification of β-Glucan

To quantify β-glucan in the shiitake extract, the β-glucan purified in Example 2 was dissolved in a sodium hydroxide aqueous solution (1125) and then precipitated (coagulated) with methanol. This procedure was repeated twice, and then the sample was washed with methanol and acetone and dried under reduced pressure (40° C., 15 hours) to give standard β-glucan containing 0.03% or less nitrogen and 1.5% or less loss on drying.

The β-glucan concentration in the shiitake extract was quantitatively determined by application of the method of Sasaki et al. (Gann, 67(2) 191-5 (1976)), using the previously purified standard β-glucan. Specifically, the shiitake extract and the standard β-glucan were prepared in a sodium hydroxide aqueous solution (2 g/dl) respectively, and a Congo red solution (10 mg/dl) and phosphoric acid (2.5 g/dl) were added thereto. Utilizing the shift of the local maximum absorption wavelength of Congo red by β-glucan, the β-glucan was quantitatively determined by measuring the absorbance at 535 nm with a spectrophotometer.

The shiitake extract quantified for β-glucan level was used to prepare a micellar shiitake extract (lecithin concentration, 0.8 mg/ml) containing β-glucan at a concentration of 0.2 mg/ml in the same manner as described in Example 1. The β-glucan solution was also subjected to emulsification treatment with lecithin to prepare a micellar β-glucan solution containing β-glucan at a concentration of 0.2 mg/ml in the same manner.

### Example 5

Inhibitory Effect of the Micellar β-Glucan on Tumor Growth

The inhibitory effects of the micellar shiitake extract, the micellar β-glucan and the α-glucan solution prepared above on tumor growth were examined in the same manner as described in Example 3. Specifically, sarcoma 180 tumor cells maintained by intraperitoneal injection in ICR mice (female, 4-week-old) were collected in the form of ascitic fluid and prepared at a density of 3×10⁷ cells/ml with physiological saline. This cell suspension was subcutaneously inoculated in a volume of 0.1 ml/mouse through a 25 G needle into a right groin of ICR mice (female, 4-week-old).

On the next day, the mice were grouped (7 mice/group) depending on their weight, and were identified, and then administrations of the micellar shiitake extract, the micellar β-glucan and the β-glucan were initiated. The extract was administered orally (one time/day) 5 times per week, and the administration was conducted 10 times in total. The dose for each administration was as follows: the sample adjusted to a β-glucan concentration of 0.2 mg/ml was given in a dose of 0.1 ml/mouse to an each 1 mg (in terms of β-glucan)/kg administration group.

The number in the brackets after “Micellar shiitake extract”, “Micellar β-glucan” and “β-Glucan”, shown in the item “Administration group” in Tables 3 and 4, is the administration dose in terms of β-glucan (unit: mg/kg). In this example, the particles (the superfine particles) subjected to superfine pulverization treatment with an emulsifier (emulsifying agent) are referred to as the micellar shiitake extract and micellar β-glucan.

TABLE 3
Tumor weight (g)

| | Day 16 | | | |
|---|---|---|---|---|
| Administration group | Average (g) | S.E. | t-test | M test |
| Non-treatment group | 3.011 | 0.323 | — | — |
| Micellar shiitake extract (1) | 1.496 | 0.343 | p < 0.01 | p < 0.01 |
| Micellar β-glucan (1) | 1.468 | 0.256 | p < 0.01 | p < 0.01 |
| β-Glucan (1) | 2.041 | 0.391 | N.S. | N.S. |

Day 16: 16th day after transplant of the tumor
t-test: Student's t-test: t-test of each group as compared with the non-treatment group was conducted.
M test (assay): Mannwhitney-U test (assay): Rank test of each group as compared with the non-treatment group was conducted.
N.S., not significant;
p < 0.01, significant.

In the groups that were administered with the micellar shiitake extract and the micellar α-glucan respectively i.e. the groups administered the superfine particles with superfine pulverization treatment, the tumor growth was inhibited gradually as compared with the non-treatment group during the administration period. On Day 16 (2nd day after the administration was finished), the tumor growth was inhibited significantly (p<0.01) in the group orally administered the micellar particles in a dose of 1 mg/kg in terms of β-glucan.

TABLE 4
Inhibitory effect on tumor growth

| | Degree of inhibition |
|---|---|
| | of tumor growth (%) |
| Administration group | Day 16* |
| Non-treatment group | — |
| Micellar shiitake extract (1) | 50.3 |
| Micellar β-glucan (1) | 51.2 |
| β-Glucan (1) | 32.2 |

*16th day after transplant of the tumor

As is evident from the results shown above, it was confirmed that the superfine particles of the present invention (micellar shiitake extract and micellar β-glucan) exhibit the desired pharmaceutical effect as compared with the conventional product (β-glucan solution).

### Example 6

Patho-Histological Investigation: Activation of Mucosal Immunity in the Intestinal Tract

The activating effect of the micellar shiitake extract on mucosal immunity in the intestinal tract was investigated in the same manner as described in Example 3. Specifically, sarcoma 180 tumor cells maintained by intraperitoneal injection in ICR mice (female, 4-week-old) were collected in the form of ascitic fluid and prepared at a density of 3×10⁷ cells/ml with physiological saline. This cell suspension was subcutaneously inoculated in a volume of 0.1 ml/mouse through a 25 G needle into a right groin of ICR mice (female, 4-week-old).

On the next day, the mice were grouped (3 mice/group) depending on their weight, and were identified, and then administrations of an emulsifier (lecithin) only, the emulsifier plus the shiitake extract (not subjected to superfine pulverization treatment [conversion into micelles]), and the micellar shiitake extract were initiated. The extract was administered orally (one time/day), 5 times per week, and the administration was conducted 10 times in total. The dose for each administration was as follows: lecithin as the emulsifier at a concentration of 2 mg/ml was given in a dose of 0.1 ml/mouse to a group administered the emulsifier; the emulsifier plus the shiitake extract were given at a concentration of 0.2 mg/ml β-glucan (lecithin concentration: 2.0 mg/ml) in a dose of 0.1 ml/mouse to a group administered the emulsifier plus the shiitake extract in a dose of 1 mg/kg in terms of β-glucan; and the micellar shiitake extract adjusted to a concentration of 0.2 mg/ml β-glucan (lecithin concentration: 2 mg/ml) was given in a dose of 0.1 ml/mouse to a group administered the micellar shiitake extract in a dose of 1 mg/kg in terms of β-glucan or in a dose of 0.3 ml/mouse to a group administered the micellar shiitake extract in a dose of 3 mg/kg in terms of β-glucan.

On the second day after completion of the administrations, small intestines with Peyer patches were removed from each group and normal mice (3 animals) and then fixed in 10% formalin and embedded in paraffin to prepare paraffin sections which were then deparaffined and stained with hematoxylin/eosin, and accumulation of mononuclear cells (immunocompetent cells) in a lamina propria mucosae in the intestinal tract was observed under a microscope.

The number in the brackets after “Shiitake extract” and “Micellar shiitake extract”, shown in the item “Administration group” in Table 5, is the administration dose in terms of β-glucan (unit: mg/kg). In this example, the particles (the superfine particles) subjected to superfine pulverization treatment with an emulsifier (emulsifying agent) are referred to as the micellar shiitake extract.

TABLE 5
Number of areas of mononuclear cell accumulation in the
lamina propria mucosae in the intestinal tract (percentage (%):
number of areas per number of villus.)

| | Percentage (%) | | |
|---|---|---|---|
| | of mononuclear | | |
| | cells accumulation | | |
| | per villus | | |
| Administration group | Average (%) | S.D. | t-test |
| Normal mice*¹ | 0.00 | 0.00 | — |
| Non-treatment group | 0.31 | 0.27 | — |
| Emulsifier only | 0.24 | 0.41 | N.S. |
| Emulsifier + shiitake extract (1)*² | 0.35 | 0.60 | N.S. |
| Micellar shiitake extract (1) | 1.33 | 0.73 | P < 0.1 |
| Micellar shiitake extract (3) | 1.34 | 0.08 | P < 0.01 |

The number of villi (100 to 300 villi/mouse) was measured under a microscope, and the number of area of mononuclear cell accumulation in the lamina propria mucosae in the intestinal tract was measured, and the percentage of number of accumulated areas per villus was calculated.
*¹Area of accumulation of mononuclear cells was not observed in the lamina propria mucosae in the intestinal tract in the normal mice.
*²The shiitake extract was merely added to the emulsifier (lecithin) and not subjected to superfine pulverization treatment (“conversion into micelles”).
t-test: Student's t-test: t-test of each group as compared with the non-treatment group was conducted.
N.S., not significant;
p < 0.1, falsely significant;
p < 0.01, significant.

Tissues of small intestines in the normal mice, the untreated group in the tumor bearing mice (non-treatment group), the group treated with the emulsifier (group administered orally 10 mg/kg lecithin), the group with the oral administration of the emulsifier plus the shiitake extract in a dose of 1 mg/kg in terms of β-glucan, and the groups administered orally the micellar shitake extract in doses of 1 mg/kg and 3 mg/kg in terms of β-glucan were observed under a microscope. As a result, the groups administered the micellar shiitake extract as compared with the non-treatment group showed accumulation of mononuclear cells (lymphocytes, macrophages) in a lamina propria mucosae in the small intestine falsely significantly (p<0.1) in the group given 1 mg/kg, and significantly (p<0.01) in the group given 3 mg/kg. In the groups other than the groups administered the micellar shiitake extract, there was no difference from the non-treatment group. Accordingly, it is considered that immune reaction is induced (activated) in the lamina propria mucosae in the intestinal tract by orally administering the micellar shiitake extract.

As is evident from the results shown above, it was confirmed that the superfine particles of the present invention (micellar shiitake extract) exhibit the desired pharmaceutical effect as compared with the conventional product (shiitake extract).

### Example 7

Activation of Systemic Immunity: Delayed Type Hypersensitive Reaction

To investigate the activation of systemic immune reaction specifically on tumor antigen, the delayed type hypersensitive reaction was evaluated. The method of evaluating the delayed type hypersensitive reaction is described.

The tumor was transplanted in the same manner as described in Example 3. Specifically, sarcoma 180 tumor cells maintained by intraperitoneal injection in ICR mice (female, 4-week-old) were collected in the form of ascitic fluid and prepared at a density of 3×10⁷ cells/ml with physiological saline. This cell suspension was subcutaneously inoculated in a volume of 0.1 ml/mouse through a 25 G needle into a right groin of ICR mice (female, 4-week-old).

On the next day, the mice were grouped (7 mice/group) depending on their weight, and were identified, and then administration of the micellar shiitake extract, the micellar β-glucan and the β-glucan solution were initiated. Each sample was administered orally (one time/day) 5 times per week, and the administration was conducted 9 times in total. The dose for each administration was as follows: each of the micellar shiitake extract and the micellar β-glucan, adjusted to a concentration of 0.2 mg/ml in terms of β-glucan (lecithin concentration: 0.8 mg/ml), was given in a dose of 0.1 ml/mouse to a group administered that in a dose of 1 mg/kg in terms of β-glucan. The β-glucan solution, adjusted to 0.2 mg/ml in a concentration of β-glucan, was administered in a dose of 0.1 ml/mouse to a group administered the solution in a dose of 1 mg/kg β-glucan.

A delayed type hypersensitivity (DTH) test was performed with the seven mice from the following groups: the non-treatment group, the group administered the micellar shiitake extract, the group administered the micellar β-glucan, the group administered the β-glucan solution, and 3 normal mice. Specifically, on the 9th day after the tumor inoculation (that is, on the 8th day after the administration was initiated), 50 μl physiological saline was administered as the control into the right foot pad, while 50 μl tumor antigen solution obtained from sarcoma 180 cells by a 3 M KCl solubilization method or a freezing and thawing method (cellular suspension at a density of 5×10⁷ cells/ml was treated) was administered into the left foot pad, and 24 hours later, the thickness of each of the right foot pad and the left foot pad was measured, and the swelling of the foot was calculated from the following equation, to evaluate the DTH reaction.

Swelling of foot (mm)=thickness of left foot pad (mm)−thickness of right food pad (mm)

The number in the brackets after “Micellar shiitake extract”, “Micellar β-glucan” and “β-Glucan solution”, shown in the item “Administration group” in Table 6, is the dose of the administered sample in terms of β-glucan (unit: mg/kg). In this example, the superfine particles subjected to superfine pulverization treatment with an emulsifier (emulsifying agent) are referred to as the micellar shiitake extract and the micellar β-glucan.

TABLE 6
Delayed type hypersensitive reaction; swelling of foot (mm)

| | Swelling of foot (mm) | | |
|---|---|---|---|
| Administration group | Average (mm) | S.D. | t-test |
| Normal mice*¹ | 0.08 | 0.05 | — |
| Non-treatment group | 0.01 | 0.05 | — |
| Micellar shiitake extract (1) | 0.23 | 0.25 | p < 0.05 |
| β-Glucan solution (1) | 0.08 | 0.11 | N.S. |
| Micellar β-glucan (1) | 0.17 | 0.08 | p < 0.01 |

*¹The normal mice did not cause DTH reaction because they did not undergo tumor inoculation (antigen sensitization).
t-test: Student's t-test: t-test of each group as compared with the non-treatment group was conducted.
N.S., not significant;
p < 0.05, significant;
p < 0.01, significant

As shown in Table 6, the swelling of foot pads in the normal mice, the non-treatment group, and the group administered the β-glucan solution was scarcely observed, while significant swelling of foot pads in the group administered the micellar shiitake extract and the group administered the micellar β-glucan was observed significantly as compared with the non-treatment group, and it was thus confirmed that the delayed type hypersensitive reaction was induced. These results indicate that systemic immune reaction against the tumor antigen can be induced by oral administration of the micellar shiitake extract and the micellar β-glucan.

### Example 8

Anti-Allergic Effect

The anti-allergic effect of the micellar shiitake extract was examined using NC mice, i.e. model mice with atopic dermatitis. Specifically, twenty 8-week-old male NC mice were sensitized by applying 150 μl/mouse antigen; picryl chloride (5% (w/v)) prepared in ethanol and acetone (4:1) onto their abdomens and foot pads, and from the fourth day after the sensitization, an inducing antigen; picryl chloride (0.8% (w/v)) prepared in olive oil was applied once per week in an amount of 150 μl/mouse for 6 weeks onto the back and ears (internal and external sides). The administration was initiated from the previous day of sensitization with the antigen and conducted once daily for 46 days. The amount of each sample administered was as follows: As the solvent control, physiological saline was orally administered in an amount of 0.1 ml/mouse into the control group (10 mice). The micellar shiitake extract, adjusted to a concentration of 0.2 mg/ml in terms of β-glucan, was orally administered in an amount of 0.1 ml/mouse. This dose in each administration corresponds to a dose of 1 mg/kg in terms of β-glucan.

For evaluation of the anti-allergic effect, sensitization with the antigen was conducted once every week, the body weight was measured, and for the degree of dermatitis, (1) pruritus/itching, (2) erythemalhemorrhage, (3) edema, (4) excoriation/erosion and (5) scaring/dryness were observed and evaluated (evaluation point: 0, asymptomatic; 1, slight; 2, moderate; 3, severe). On the 29th and 46th days after sensitization with the antigen, blood was collected from the orbital vein, serum was obtained therefrom, and the immunoglobulin E (IgE) level in the serum was measured by ELISA.

Results: On the 46th day after the sensitization, the IgE level was at least 5.0 μg/ml in all 10 mice in the control group, while the IgE level in 4 mice out of 10 mice in the group administrated the micellar shiitake extract was at least 5.0 μg/ml, but the other 6 mice indicated an IgE level of less than 5.0 μg/ml, thus showing that the increase of the IgE level was significantly suppressed (Fisher's probability test: p<0.05). Further, on the 46th day after the sensitization, the total dermatitis score was 9 or more in 9 mice out of 10 mice in the control group, while the total dermatitis score was 9 or more in 5 mice out of 10 mice in the group administrated the micellar shiitake extract, and the other 5 mice indicated a total dermatitis score of less than 9, showing that the dermatitis was suppressed as compared with the control group.

As is evident from the results shown above, it was confirmed that the superfine particles of the present invention (micellar shiitake extract) exhibit the desired pharmaceutical effect (anti-allergic effect).

### Example 9

Anti-Diabetic Effect: Inhibitory Effect on Increase in Blood Sugar Levels

To investigate the effect of the micellar shiitake extract on diabetes, db/db mice, i.e. model mice with type II diabetes were used to examine the effect on increase in blood sugar levels. Specifically, 5-week-old male db/db mice were divided into 2 groups each consisting of 9 mice, and one group were allowed water ad libitum as the control group, while the other group was given micellar shiitake extract-containing water ad libitum until the mice were 12-week-old. The concentration of the micellar shiitake extract was 0.01 mg/ml in terms of α-glucan (lecithin concentration: 0.1 mg/ml). The amount of water drunk was measured every week, to calculate the amount of orally ingested β-glucan.

Blood was collected from the orbital vein every week to enable evaluation of the effect of the administration. Blood sugar levels were measured and the inhibitory effect on increase in sugar blood levels was examined. The amount of orally ingested β-glucan was in the range of from 0.15 mg/mouse/day to 0.36 mg/mouse/day in the 6- to 12-week-old mice.

In the 8-week-old mice, the blood sugar levels in the control group were 504.11±50.03 mg/dl, while the blood sugar levels in the group administered the micellar shiitake extract were 406.22±75.55 mg/dl, indicating that the blood sugar levels were significantly prevented from increasing (Student's t-test: p<0.01).

As is evident from the results shown above, it was confirmed that the superfine particles of the present invention (micellar shiitake extract) exhibit the desired pharmaceutical effect (inhibitory effect on increase in blood sugar levels in diabetes).

Numerous modifications and variations on the present invention are possible in light of the above teachings. It is, therefore, to be understood that within the scope of the accompanying claims, the invention may be practiced otherwise than as specifically described herein.

### EFFECT OF INVENTION

The present invention provides an immune activator and an immune regulator that can improve an animal, particularly human, immunocompetence. Accordingly, the present invention is particular useful as a pharmaceutical composition, food and drink (health foods, functional foods etc.) in that such compositions have excellent immune activating action and/or immune regulating action.

The present invention also provides a novel substance (or a novel composition) usable as an active (effective) ingredient for such excellent products, specifically superfine particles of a component selected from a component derived from a mushroom and β-glucan, for example, superfine particles of an extract of a mushroom, preferably a water extract thereof treated with a dispersant (dispersion), particularly a micellar solution thereof obtained by treatment with an emulsifier (emulsifying agent).

Also provided by the present invention is a method of activating immunity or a method of regulating immunity for medical treatment, prophylaxis (prevention) of various diseases, a use of the superfine particles for the immune activator or the immune regulator; or a use of the superfine particles for the antitumor agent, the anti-infective agent, the antiviral agent, the anti-autoimmune disease agent, the anti-diabetes agent and the anti-allergy agent, as well as the pharmaceutical preparations for digestive organ diseases (therapeutic agents for irritable bowel syndrome (IBS), inflammatory bowel disease (IBD), constipation, diarrhea and the like) and the like, and a use thereof for production of pharmaceutical products (preparations).

According to the present invention, there can be further provided a process for producing a mushroom-derived component and β-glucan as an efficacious ingredient by easy production means, particularly the superfine particles of the active ingredient having the immune activating action and/or the immune regulating action (or a composition comprising the same), which can produce the above superfine particles or composition. As a result, a pharmaceutical composition and a food and drink (food and/or drink) (health foods, functional foods etc.) etc. utilizing the active ingredient can be industrially and easily produced.

Accordingly, the present invention is extremely useful in industry, particularly in many fields such as medical practices (medical treatments), pharmaceutical products (preparations), foods etc.

1. A composition comprising superfine particles of a component selected from the group consisting of β-glucan and a component derived from a mushroom.

2. The composition according to claim 1, wherein said component is a component derived from a mushroom.

3. The composition according to claim 2, wherein the component derived from a mushroom is an extract of a mushroom.

4. The composition according to claim 3, wherein the extract of a mushroom is a water extract of a mushroom.

5. The composition according to claim 2, wherein the component derived from a mushroom is β-glucan or contains β-glucan.

6. The composition according to claim 1, wherein said component is β-glucan.

7. The composition according to claim 6, wherein the β-glucan is obtained from a source other than a mushroom.

8. The composition according to claim 7, wherein said source is selected from the group consisting of a yeast, a fungi, a bacterium, and a plant.

9. The composition according to claim 1, wherein the component forms aggregates in an aqueous solution.

10. The composition according to claim 9, wherein the aggregates have a particle diameter of at least 50 μm.

11. The composition according to claim 1, wherein the superfine particles have an average particle diameter of 10 μm or less, as determined in the form of a dispersion in water.

12. The composition according to claim 11, wherein the superfine particles have an average particle diameter of 1 μm or less.

13. The composition according to claim 11, wherein the superfine particles have an average particle diameter of 0.01 to 1 μm.

14. The composition according to claim 1, wherein the superfine particles have an average particle diameter of 10 μm or less and wherein the superfine particles are obtained by mixing a dispersant with an aqueous solution containing a component selected from the group consisting of β-glucan and a component derived from a mushroom.

15. The composition according to claim 14, wherein the superfine particles have an average particle diameter of 1 μm or less and wherein the superfine particles are obtained by further fine pulverizing treatment.

16. The composition according to claim 14, wherein the aqueous solution contains an extract of a mushroom obtained by filtering a water extract of a mushroom or a hot-water extract of a mushroom.

17. The composition according to claim 16, wherein the extract contains aggregates obtained by filtering a water extract of a mushroom or a hot-water extract of a mushroom and then concentrating and/or cooling the filtrate.

18. The composition according to claim 14, wherein the aqueous solution comprises β-glucan.

19. The composition according to claim 14, wherein the dispersant is an emulsifier.

20. The composition according to claim 19, wherein the emulsifier is lecithin.

21. The composition according to claim 1, further comprising a dispersant.

22. The composition according to claim 21, wherein the dispersant is mixed with an aqueous solution containing a component selected from the group consisting of β-glucan and a component derived from a mushroom such that the ratio by weight of the dispersant to the whole sugar (1) contained in the aqueous solution is 100 at most.

23. The composition according to claim 21, wherein the dispersant is an emulsifier.

24. The composition according to claim 23, wherein the emulsifier is lecithin.

25. The composition according to claim 1, wherein the superfine particles are in the form of micelles.

26. The composition according to claim 1, further comprising a pharmaceutically acceptable carrier or excipient.

27. An immune activator or an immune regulator comprising the composition described in claim 1.

28. An agent selected from the group consisting of an antitumor agent, an anti-infective agent, an antiviral agent, an anti-autoimmune disease agent, an anti-diabetes agent, an anti-allergy agent, an a pharmaceutical preparation for digestive organ diseases, a therapeutic agent for irritable bowel syndrome, a therapeutic agent for inflammatory bowel disease, a therapeutic agent for constipation, and a therapeutic agent for diarrhea, wherein said agent comprises the composition described in claim 1.

29. A food or drink comprising the composition described in claim 1.

30. The food or drink according to claim 29, which comprises the superfine particles of the composition are in an amount of 0.01 to 80% by weight based on the whole sugar.

31. A superfine particle-containing composition comprising an aqueous solution of the composition described in claim 1 dispersed therein.

32. A pharmaceutical composition comprising the superfine particle-containing composition described in claim 31 and further comprising a pharmaceutically acceptable carrier or excipient.

33. A food or drink comprising the superfine particle-containing composition described in claim 31.

34. The food or drink according to claim 33, which comprises the superfine particle-containing composition in an amount of 0.05 to 5% by weight based on the whole sugar.

35. The superfine particle-containing composition according to claim 31, which comprises 1 to 20000 mg sugar and 1 to 20000 mg dispersant every 100 g of the composition.

36. A process for producing superfine particles comprising superfine pulverizing a component selected from the group consisting of β-glucan and a component derived from a mushroom.

37. The process according to claim 36, wherein the component derived from a mushroom is an aqueous extract obtained in a step of extraction from a mushroom with water.

38. The process according to claim 36, wherein said superfine pulverizing includes preparing particles having an average particle diameter of 10 μm or less by mixing a dispersant with an aqueous solution containing a component selected from the group consisting of β-glucan and a component derived from a mushroom.

39. The process according to claim 38, wherein the aqueous solution contains an extract of a mushroom obtained by filtering a water extract of a mushroom or hot-water extract of a mushroom.

40. The process according to claim 36, wherein said superfine pulverizating includes preparing particles having an average particle diameter of 1 μm or less.

41. The process according to claim 40, wherein said preparing particles having an average particle diameter of 1 μm or less comprising treating the particles with a high-pressure emulsifier.

42. A process for producing a composition containing superfine particles comprising superfine pulverizing a component selected from the group consisting of β-glucan and a component derived from a mushroom.

43. The process according to claim 42, wherein said superfine pulverizing includes preparing particles having an average particle diameter of 10 μm or less by mixing a dispersant with an aqueous solution containing a component selected from the group consisting of β-glucan and a component derived from a mushroom.

44. The process according to claim 43, wherein the aqueous solution contains an extract of a mushroom obtained by filtering a water extract of a mushroom or hot-water extract of a mushroom.

45. The process according to claim 42, wherein said superfine pulverizating includes preparing particles having an average particle diameter of 1 μm or less.

46. The process according to claim 45, wherein said preparing particles having an average particle diameter of 1 μm or less comprising treating the particles with a high-pressure emulsifier.

47. The process according to claim 43, wherein the component derived from a mushroom is an aqueous extract obtained in a step of extraction from a mushroom with water.

48. A method of activating or regulating immunity comprising administering to a subject in need thereof a composition described in claim 1.

49. The method according to claim 48, wherein the subject in need thereof has a condition selected from the group consisting of a tumor, an infection, a viral infection, an autoimmune disease, diabetes, an allergy, a digestive organ disease, irritable bowel syndrome, inflammatory bowel disease, constipation, and diarrhea.

50. The method according to claim 48, wherein the composition further comprises a pharmaceutically acceptable carrier or excipient.

51. The method according to claim 48, wherein the composition is admixed with a food and drink prior to administration to said subject in need thereof.
